(19) United States
(12) Patent Application Publication
(10) Pub. No.: US 2025/0043222 A1
(43) Pub. Date: Feb. 6, 2025

(54) BIOREACTORS, SYSTEMS COMPRISING THE SAME AND METHODS OF USE THEREOF

(71) Applicant: Pairwise Plants Services, Inc., Durham, NC (US)

(72) Inventor: Douglas Lee Heckart, Wake Forest, NC (US)

(21) Appl. No.: 18/718,124

(22) PCT Filed: Dec. 15, 2022
(86) PCT No.: PCT/US2022/081611

§ 371 Date: Jun. 10, 2024

Related U.S. Application Data
(60) Provisional application No. 63/289,826, filed on Dec. 15, 2021.

(51) Int. Cl. C12M 1/00; C12M 1/12; C12M 1/34; C12N 1/20; C12N 5/04

(52) U.S. Cl. CPC C12M 23/34; C12M 23/58; C12M 29/04; C12M 29/06; C12M 29/14; C12M 37/02; C12M 41/06; C12M 41/12; C12M 41/34; C12M 41/40; C12N 1/20; C12N 5/04

(57) ABSTRACT

Described herein are bioreactors and systems comprising the same (e.g., a bioreactor assembly). Also described herein are methods of using the bioreactors and systems.

### FIELD

The present invention relates to bioreactors and systems comprising the same (e.g., a bioreactor assembly). Methods of use of the bioreactors and systems are also provided herein.

### BACKGROUND

Overgrowth of bacteria (e.g., Agrobacterium) and/or fungi such as during plant transformation can be problematic to plant tissues. Accordingly, new systems and/or methods are needed to address these problems.

### SUMMARY OF THE INVENTION

One aspect of the present invention is directed to a method of co-culturing a plant explant and bacteria, the method comprising: culturing a plant explant comprising (e.g., inoculated with) bacteria in a bioreactor vessel, the bioreactor vessel comprising an internal cavity, a permeable member that divides the internal cavity into upper and lower compartments, a dispensing inlet positioned to feed the upper compartment, and a drainage outlet configured to drain the lower compartment, wherein the plant explant comprising the bacteria is present on the permeable member; spraying media onto the plant explant comprising the bacteria via the dispensing inlet, wherein, following spaying, excess media travels through the permeable member to the lower compartment to provide waste media in the lower compartment; and removing the waste media via the drainage outlet, thereby co-culturing the plant explant and the bacteria.

Another aspect of the present invention is directed to a bioreactor assembly, comprising: a bioreactor vessel having an internal cavity and a permeable member that divides the internal cavity into upper and lower compartments, the bioreactor vessel further including a dispensing inlet positioned to feed the upper compartment and a drainage outlet configured to drain the lower compartment; a medium tank fluidly connected to the dispensing inlet; a drain fluidly connected to the drainage outlet; and a positive pressure source operatively associated with the medium tank and the dispensing inlet configured to force medium from the medium tank through the dispensing inlet and into the upper compartment.

### BRIEF DESCRIPTION OF THE FIGURES

FIG. 1 is a flow chart illustrating operations according to embodiments of the invention.

FIG. 2 is a schematic diagram illustrating a bioreactor assembly according to embodiments of the invention.

FIG. 3 is a schematic diagram illustrating a bioreactor assembly according to alternative embodiments of the invention.

FIG. 4 is a schematic diagram illustrating a bioreactor assembly according to additional embodiments of the invention.

FIG. 5 is a schematic diagram illustrating a bioreactor assembly according to further embodiments of the invention.

FIG. 6 is a schematic diagram illustrating a bioreactor assembly according to still further embodiments of the invention.

### DETAILED DESCRIPTION OF EXEMPLARY EMBODIMENTS

The present invention is now described more fully hereinafter in which embodiments of the invention are described. This invention may, however, be embodied in many different forms and should not be construed as limited to the embodiments set forth herein; rather these embodiments are provided so that this disclosure will be thorough and complete and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a,” “an” and “the” are intended to include the plural forms as well, unless the context clearly indicates otherwise.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description of the invention herein is for the purpose of describing particular embodiments only and is not intended to be limiting of the invention. All publications, patent applications, patents and other references mentioned herein are incorporated by reference in their entirety. In case of a conflict in terminology, the present specification is controlling.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed.

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

Recitation of ranges of values herein are merely intended to serve as a shorthand method of referring individually to each separate value falling within the range, unless otherwise indicated herein, and each separate value is incorporated into the specification as if it were individually recited herein. For example, if the range 10 to 15 is disclosed, then 11, 12, 13, and 14 are also disclosed.

As used herein, the terms “increase,” “increasing,” “enhance,” “enhancing,” “improve” and “improving” (and grammatical variations thereof) describe an elevation of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more such as compared to another measurable property or quantity (e.g., a control value).

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 30%, 35%, 40%, 45%, 50%, 55%, 60%, 65%, 70%, 75%, 80%, 85%, 90%, 95%, 97%, 98%, 99%, or 100% such as compared to another measurable property or quantity (e.g., a control value). In some embodiments, the reduction can result in no or essentially no (i.e., an insignificant amount, e.g., less than about 10% or even 5%) detectable activity or amount.

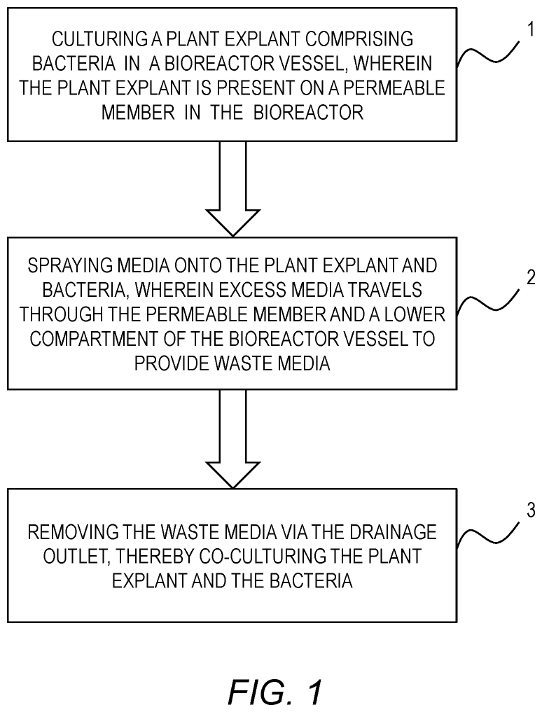

Referring now to the figures, FIG. 1 is a flow chart illustrating a method of co-culturing a plant explant and bacteria according to embodiments of the invention. The method includes: culturing a plant explant comprising (e.g., inoculated with) bacteria in a bioreactor vessel, the bioreactor vessel comprising an internal cavity, a permeable member that divides the internal cavity into upper and lower compartments, a dispensing inlet positioned to feed the upper compartment, and a drainage outlet configured to drain the lower compartment, wherein the plant explant comprising the bacteria is present on the permeable member (Block 1 of FIG. 1); spraying media onto the plant explant comprising the bacteria via the dispensing inlet, wherein, following spaying, excess media travels through the permeable member to the lower compartment to provide waste media in the lower compartment (Block 2 of FIG. 1); and removing the waste media via the drainage outlet, thereby co-culturing the plant explant and the bacteria (Block 3 of FIG. 1). The method is discussed in more detail below.

A plant explant (e.g., a plant explant comprising bacteria) may be cultured using methods known in the art. For example, culturing a plant explant may include exposing the plant explant to certain conditions (e.g., light, dark, nutrients, humidity, etc.) for a period of time (e.g., about 1, 2, 3, 4, or 5 day(s) to about 6, 7, 8, 9, 10, 11, 12, 13, 14, or more days). In some embodiments, culturing a plant explant comprises providing temperature and/or light conditions sufficient to maintain and/or grow the plant explant. In some embodiments, culturing a plant explant includes spraying media onto the plant explant. The media may include an antibiotic, a growth hormone (e.g., a plant growth hormone, and/or a nutrient). In some embodiments, solid media (e.g., solid growth media) is not used in a method and/or culturing step of the present invention. In some embodiments, a bioreactor vessel is devoid of a solid media (e.g., solid growth media).

The bioreactor vessel may be any vessel known to be suitable for culturing a plant explant and/or suitable for conducting and/or carrying out transformation of a plant explant. The bioreactor vessel may be configured to provide and sustain certain conditions that are suitable or desirable for explant culturing, such as temperature, humidity, light, and the like. The bioreactor vessel includes an internal cavity within which culturing of an explant and/or a transformation step can occur, and may provide a sterile environment for the explant.

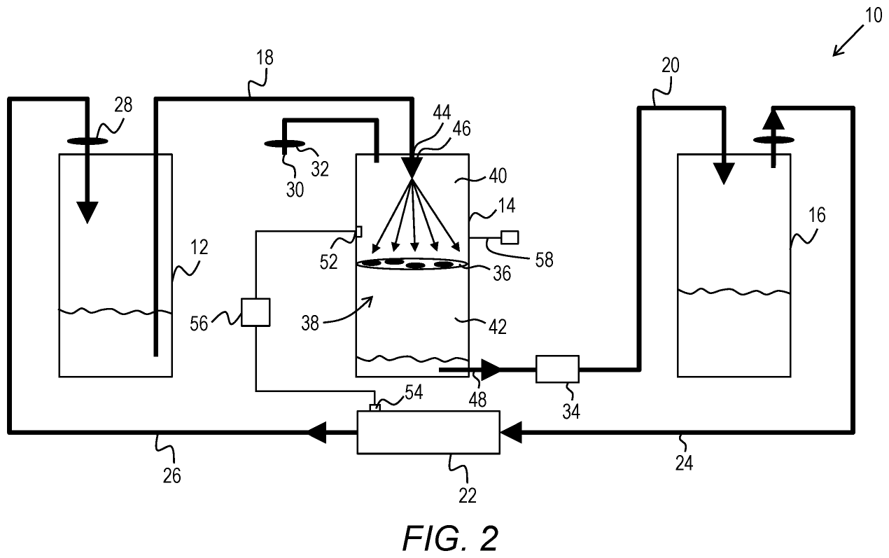

As described in detail below in connection with FIGS. 2-6, the bioreactor vessel has an internal cavity fed by a dispensing inlet and drained by a drainage outlet. A permeable member, such as a screen, mesh, perforated plate or panel, or the like is positioned within the cavity of the bioreactor to divide the cavity into upper and lower compartments. The dispensing inlet feeds into the upper compartment, and the drainage outlet drains from the lower compartment. As used herein, the “upper compartment” encompasses the volume within the cavity above and including the permeable member, and the “lower compartment” encompasses the volume within the cavity below the permeable member. The permeable member may be sealed to the walls of the bioreactor vessel, such that medium can also pass through the permeable member, or may be mounted within the cavity so that a gap is present between the permeable member and the walls of the bioreactor vessel that permits the passage of medium.

The permeable member is configured so that plant explant is supported by and remains on the permeable member, but medium may flow through the permeable member. As an example, holes, apertures, openings, pores or the like in the permeable member may have a pore size of about 10 microns or more.

The spraying of media onto the plant explant (Block 2) may be performed in any manner suitable for conveying the media onto the explant. As used herein, “spraying” is intended to encompass any manner of conveying liquid medium onto the explant in a multi-stream form, including as examples a spray, mist, atomized spray, and/or aerosol. The bioreactor inlet may comprise a nozzle that receives liquid media and distributes the medium within the bioreactor as a spray.

In some embodiments, the spraying of medium onto the plant explant is achieved by the application of positive pressure to a source of medium that forces the medium into the bioreactor. Positive pressure is typically provided by a pump or similar device fluidly connected with the medium source.

In some embodiments, spraying is performed intermittently. Spraying may be conducted at a frequency and in an amount such that the explant remains damp (e.g., damp during culturing and/or during a method of the present invention). Exemplary spraying parameters and conditions include spraying media for about 1, 5, or 10 seconds to about 15, 20, or 30 seconds every hour, optionally every hour during the time period of culturing the explant. For example, an explant may be sprayed for about 10 seconds every hour during culturing of the explant. The volume of sprayed media during a single, continuous session (e.g., cycle) may have a volume of about 10, 15, or 15 mL to about 20, 25, 30, 40, 50, 60, 70, 80, 90, or 100 mL. The volume of media sprayed during a single cycle may vary based on the area of the explant and/or area of the permeable member. In some embodiments, about 20 mL may be sprayed during a single cycle to cover an area of about 70 cm² and/or about 50 mL may be sprayed during a single cycle to cover an area of about 400 cm².

In some embodiments, spraying media onto a plant explant may result in washing contaminants and/or metabolites (e.g., bacteria and/or phenolics) off the plant explant, which may improve plant explant health, survival, and/or transformation rate. Cutting of plant tissue can trigger the plant material to produce phenolics, which can be a defense response to protect against pathogen infection and thereby inhibit Agrobacterium infection during a transformation method. When a plant explant is plated on solid medium, phenolics are released into the solid medium; the explant sits on this medium, and the released phenolics cause the plant tissue to turn brown (e.g., sustain damage and/or die). In some embodiments, a method of the present invention may increase the survival rate of plant explants comprising the bacteria compared to a control method (e.g., a method of culturing plant explants comprising bacteria on a solid media). In some embodiments, a method of the present invention increases the processing rate (i.e., amount of plant material processed (e.g., cultured) over a period of time) of plant explants comprising bacteria compared to the processing rate of plant explants comprising bacteria for a control method (e.g., a method of culturing plant explants comprising bacteria on a solid media). A method of the present invention may improve throughput and/or the amount of plant material transformed.

After spraying, some of the medium may remain on the explant (e.g., as a thin liquid layer coating the explant material), and some of the medium passes through the permeable member and into the lower compartment of the bioreactor. Also, as the explant resides on the permeable member after spraying, more of the medium will tend to drip through the permeable member into the lower compartment. Thus, waste medium (comprising both medium that passes into the lower compartment during spraying and medium that drips from the explant through the permeable member after spraying) collects in the lower compartment. The waste medium is removed (Block 3 of FIG. 1) through the drainage outlet.

The waste medium can be removed via any manner known to be suitable for removing liquid from a bioreactor vessel. For example, the waste medium may be drawn through the drainage outlet via the application of negative pressure (i.e., suction) to the lower compartment through the drainage outlet (for example, with a suction pump). Alternatively, the waste medium may be removed via simple gravimetric drainage. Oher techniques may be known to those of skill in this art.

Waste medium may be removed simultaneously with the spraying of fresh medium into the bioreactor, or may be removed before and/or after the spraying of fresh medium.

Culturing of the explant can benefit from the removal of waste medium because this may prevent pathogen (e.g., bacteria and/or fungi) growth in the bioreactor vessel.

Typically, the explant remains on the permeable member for a number of “cycles,” wherein each cycle includes the spraying of fresh medium and the removal of waste medium. The number and duration of cycles may vary based on the identity of the explant and/or culturing bacteria. Exemplary cycle durations include about 10 seconds to about 10 minutes. Exemplary numbers of cycles include about 12 to about 72 or 100 or more. In some embodiments, a method of the present invention and/or culturing step is carried out for about 1, 2, 3, 4, 5, 6, 7, 8, 9, 10, 11, 12, 13, or 14 days or more with about 12, 24, 36, or more cycles per day.

In some embodiments, prior to Block 1 of FIG. 1, a plant explant may be contacted with a bacteria or vice versa to thereby provide a plant explant comprising the bacteria. The bacteria may be present on a surface of the plant explant and/or may be within the plant explant. In some embodiments, the method comprises inoculating a plant explant with a bacteria, which may allow for a polynucleotide to be introduced into a cell of the plant explant. Contacting of a plant explant and bacteria may occur outside a bioreactor vessel such that a plant explant comprising the bacteria may be placed on a permeable member in bioreactor vessel. Alternatively, contacting of a plant explant and bacteria may occur inside a bioreactor vessel such that the bacteria is sprayed onto the explant via the dispensing inlet of the bioreactor vessel to thereby provide the plant explant comprising the bacteria. In some embodiments, the bacteria may be present in media that is sprayed onto the explant via the dispensing inlet of the bioreactor vessel. In some embodiments, a plant explant comprising bacteria may be contacted with additional bacteria that are the same as or different than the bacteria present on and/or in the explant comprising bacteria. Accordingly, a method of the present invention may comprise introducing a polynucleotide into a cell of the plant explant using the bacteria contacted to the plant explant.

“Introducing,” “introduce,” “introduced” (and grammatical variations and derivatives thereof) in the context of a polynucleotide and/or polypeptide means presenting a nucleotide sequence of interest (e.g., polynucleotide, a nucleic acid construct, and/or a guide nucleic acid) and/or polypeptide of interest, respectively, to a host organism or cell of said organism (e.g., host cell; e.g., a plant cell) in such a manner that the nucleotide sequence and/or polypeptide, respectively gains access to the interior of a cell. Thus, for example, a polynucleotide from an Agrobacterium strain, such as A. tumifaciens or A. rhizogenes, (that may encode or comprise at least a portion of an editing system) may be introduced into a cell of an organism (e.g., a eukaryote such as a human or plant), thereby transforming the cell with the polynucleotide. In some embodiments, a nucleic acid construct of the invention encoding a CRISPR-Cas effector protein, a guide nucleic acid, and optionally a deaminase (e.g., a cytosine deaminase and/or adenine deaminase) or optionally a reverse transcriptase may be introduced into a cell of an organism, thereby transforming the cell with the CRISPR-Cas effector protein, guide nucleic acid, and deaminase or reverse transcriptase. In some embodiments, a guide nucleic acid and/or a polypeptide comprising a CRISPR-Cas effector protein may be introduced into a cell of an organism, optionally wherein the CRISPR-Cas effector protein and guide nucleic acid may be comprised in a complex (e.g., a ribonucleoprotein) that is introduced into the cell. In some embodiments, the organism is a eukaryote (e.g., a plant or mammal such as a human).

The term “transformation” as used herein refers to the introduction of a heterologous nucleic acid into a cell. Transformation of a cell may be stable or transient. Thus, in some embodiments, a host cell or host organism may be stably transformed with a polynucleotide/nucleic acid molecule of the invention. In some embodiments, a host cell or host organism may be transiently transformed with a nucleic acid construct of the invention.

“Transient transformation” in the context of a polynucleotide means that a polynucleotide is introduced into a cell (e.g., by a transformation and/or transfection approach) and does not integrate into the genome of the cell; thus, the cell is transiently transformed with the polynucleotide. A nucleic acid that is “transiently expressed” as used herein refers to a nucleic acid that has been introduced into a cell and the nucleic acid is not integrated into the genome of the cell, thereby the cell is transiently transformed with the nucleic acid.

By “stably introducing” or “stably introduced” in the context of a polynucleotide introduced into a cell (e.g., by a transformation and/or transfection approach) is intended that the introduced polynucleotide is stably incorporated into the genome of the cell, and thus the cell is stably transformed with the polynucleotide. A nucleic acid that is “stably expressed” as used herein refers to a nucleic acid that has been introduced into a cell and the nucleic acid is integrated into the genome of the cell, thereby the cell is stably transformed with the nucleic acid.

“Stable transformation” or “stably transformed” as used herein means that a nucleic acid molecule is introduced into a cell (e.g., by a transformation and/or transfection approach) and integrates into the genome of the cell. As such, the integrated nucleic acid molecule is capable of being inherited by the progeny thereof, more particularly, by the progeny of multiple successive generations. “Genome” as used herein includes the nuclear and the plastid genome, and therefore includes integration of the nucleic acid into, for example, the chloroplast or mitochondrial genome. Stable transformation as used herein can also refer to a transgene that is maintained extrachromasomally, for example, as a minichromosome or a plasmid.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant). Stable transformation of a cell can be detected by, for example, a Northern blot hybridization assay of RNA of the cell with nucleic acid sequences which specifically hybridize with a nucleotide sequence of a transgene introduced into a host organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art. A method of the present invention may be part of a transformation method. In some embodiments, a transformation method is carried out and/or performed in a bioreactor vessel and/or bioreactor assembly of the present invention.

Spraying and removing steps of the present invention may be carried out (e.g., intermittently carried out) during a culturing step of the present invention. In some embodiments, a method of the present invention is carried out in a closed system in that the system does not expose the plant explant comprising the bacteria to the environment external to the bioreactor vessel and/or system (e.g., bioreactor assembly). One or more (e.g., 1, 2, 5,10, 20, 50, or more) bioreactor vessels may be used and/or connected in a method and/or system of the present invention.

A method of the present invention may be carried out for about 1, 2, 3, 4, 5, 6,7, 8, 9, 10, 11, 12, 13, or 14 days or more. In some embodiments, a method of the present invention may be carried out for a period of time (e.g., about 1 to about 7 or 14 days or more) without removing an explant from the bioreactor vessel and/or without exposing an explant to the external environment once the explant has been placed in the bioreactor vessel. A method of the present invention may provide for fresh media and/or antibiotics to contact a plant explant, which may reduce or prevent contaminates and/or bacteria growth in a bioreactor vessel of the present invention.

Referring now to FIG. 2, a bioreactor assembly that may be suitable for carrying out the methods discussed above is schematically illustrated therein and designated broadly at 10. The assembly 10 includes a medium tank 12 filled with medium (e.g., a growth media), a bioreactor vessel 14, and a drainage receptacle 16. A supply line 18 fluidly connects the medium tank 12 with the bioreactor vessel 14. A drain line 20 fluidly connects the bioreactor vessel 14 with the drainage receptacle 16. A combination suction/pressure pump 22 is fluidly connected with drainage receptacle 16 via an air line 24 and with the medium tank 12 via an air line 26. A filter 28 is located on the air line 26 adjacent the medium tank 12. A vent 30 with a filter 32 is fluidly connected with the bioreactor vessel 14. A check valve 34 is located on the drain line 20 to prevent fluid from flowing from the drain line and/or the drainage receptacle into the bioreactor vessel 14.

As can be seen in FIG. 2, the bioreactor vessel 14 includes a screen 36 or other permeable member mounted within the cavity 38 of the bioreactor vessel 14 to divide the cavity 38 into upper and lower compartments 40, 42. At its upper end, the bioreactor vessel 14 includes an inlet 44 fed by the supply line 18. A spray nozzle 46 is fluidly connected with the supply line 18 and is positioned within the bioreactor vessel 14 (in this instance, near the upper end of the upper compartment 40). The spray nozzle 46 is located and configured to spray medium (in some embodiments, in the form of a mist, atomized spray, and/or aerosol) into the upper compartment 40 and onto any plant material residing on the screen 36. An outlet 48 is located near the lower end of the lower compartment 42 and feeds the drainage line 20.

The air line 26 and the supply line 18 are routed to and from the medium tank in a manner to render the medium tank 12 airtight. Similarly, the drainage line 20 and the air line 24 are routed to and from the drainage receptacle in a manner to render the drainage receptacle 16 airtight. The pump 22 is configured to create negative pressure (i.e., suction) within the drainage tank to draw gas (typically air) from the drainage receptacle 16. The pump 22 is also configured to create positive pressure that forces gas (again, typically air) into the medium tank 12.

One or more sensors (represented at 52 in FIG. 2) may be present in the bioreactor vessel 14 to sense operational parameters and/or conditions (such as temperature, humidity, light, oxygen concentration, carbon dioxide concentration, and the like) within the cavity 38. Additionally, the pump 22 may be connected with a timer 54 or other timing device to control the time and/or duration of operation. Either or both of the sensor(s) 52 and the pump 22 may be operatively connected to a controller 56 that controls the operation of the bioreactor assembly 10.

In addition, other agents may be introduced into the bioreactor vessel 14. For example, carbon dioxide (CO₂) may be introduced into the bioreactor vessel 10 via a CO₂ line 58.

Operation of the bioreactor assembly 10 will now be described. Plant material to be cultured is placed on the screen 36. For most of the time the plant material is on the screen, the pump 22 is idle. Under such conditions, the lower compartment may contain medium, but at a level that is below the screen 36. Parameters within the cavity 38 (such as temperature, light, humidity and the like) are controlled to create favorable conditions for plant culture and/or transformation, and may be adjusted if the sensor(s) so indicate.

Intermittently, the medium in the bioreactor vessel 14 should be refreshed. Refreshment of the medium is achieved by activating the pump 22, which generates both negative pressure (i.e., suction) to the air line 24 and positive pressure on the air line 26. Suction in the air line 24 generates a slight vacuum within the drainage receptacle 26, which is of a sufficient magnitude to draw media from the lower compartment 42 of the bioreactor vessel 14 into the drainage line 20 and, subsequently, into the drainage receptacle 16. As such, the bioreactor vessel 14 can be cleared of all excess/waste medium in the lower compartment 42. The check valve 34 prevents any medium from returning to the bioreactor vessel 14 via the supply line 20 when the vacuum ceases.

Simultaneously, as described above, the generation of positive pressure on the air line 26 generates positive pressure in the medium tank 12. The positive pressure forces fresh medium from the medium tank 12 into and through the supply line 18 and to the nozzle 46. Medium that reaches the nozzle 46 is gently sprayed (e.g., as a mist) into the upper compartment 40 of the bioreactor vessel 14 and onto the plant matter residing on the screen 36.

When the excess/waste medium has exited the bioreactor vessel 14 and the fresh medium has been sprayed onto the plant matter, the pump 22 is deactivated. While some of the misted fresh media may drip or otherwise pass through the screen 36, the remaining misted fresh medium coats the plant material as it resides on the screen 36, thereby hydrating the plant material and/or providing nutrient(s) and/or antibiotic(s) to the plant material. Over time, much of the fresh media drips from the plant material through the screen 36 and into the lower compartment 42, where it gradually collects until the next medium replenishment cycle.

Notably, the spraying/misting of the fresh medium onto the plant material during replenishment also has the effect of washing/rinsing any spent medium remaining on the plant matter. This action can assist in removing spent medium from the plant matter, thereby removing contaminants and/or plant released stress compounds and/or preventing some of the deleterious bacterial growth described above that can result from spent medium remaining in the bioreactor vessel 14.

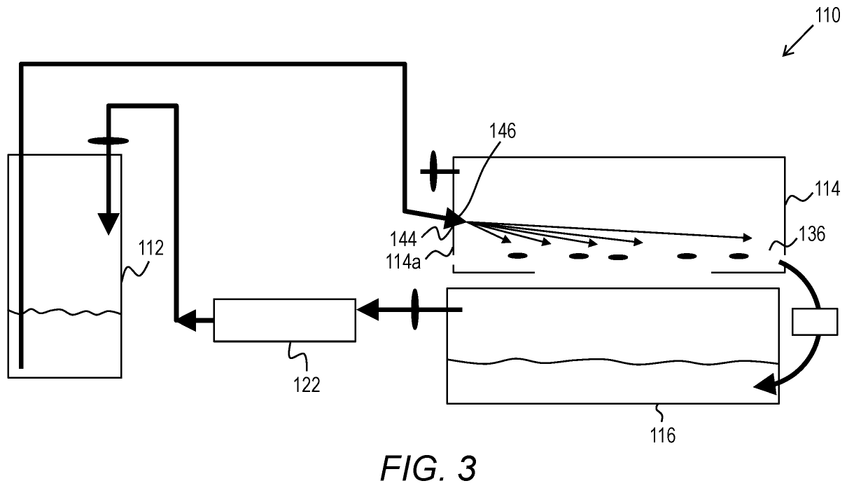

Bioreactor assemblies according to embodiments of the invention may take different forms. For example, FIG. 3 illustrates a bioreactor assembly 110 that includes the same components as the bioreactor 10 described above, with the exception that its bioreactor vessel 114 is more horizontally elongate, and its inlet 144 is located on one of the side walls 114a of the bioreactor vessel 144 rather than in the top. The nozzle 146 creates a spray/mist that travels more horizontally than that of the bioreactor assembly 10. This configuration may provide greater surface area for the screen 136 that enables a greater amount of plant matter to be transformed in each bioreactor vessel 114. The operation of the bioreactor assembly 110 follows the same sequence as described above for the bioreactor assembly 10: namely, the pump 122 creates suction in the drainage receptacle 116 that draws spent medium from the bioreactor vessel 114 into the drainage receptacle 116, and also creates positive pressure in the medium tank 112 that forces fresh medium into the bioreactor vessel 114 through the nozzle 146.

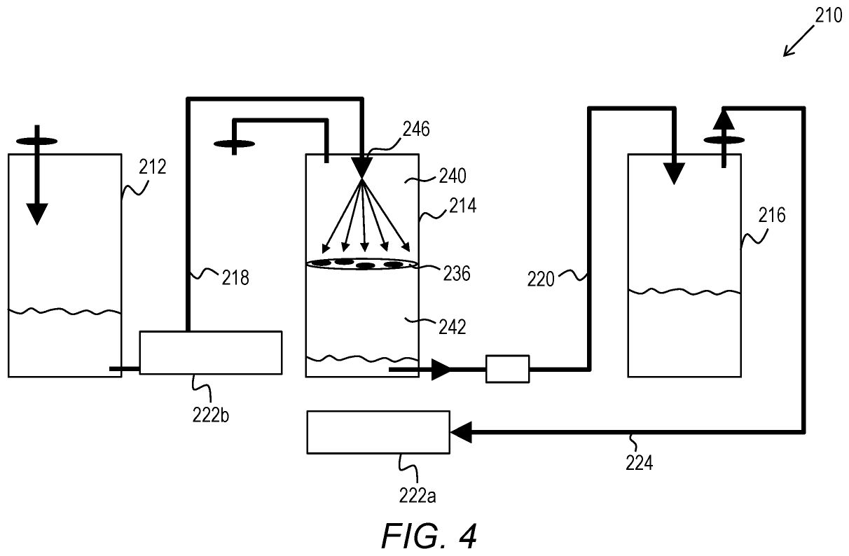

FIG. 4 illustrates another bioreactor assembly designated broadly at 210, according to embodiments of the invention. The bioreactor assembly 210 includes a medium tank 212, a bioreactor vessel 214, and a drainage receptacle 216 like those described above. However, rather than using a single pump to create negative pressure to remove spent medium and positive pressure to provide fresh medium, the bioreactor assembly 210 includes two pumps: a vacuum pump 222a fluidly connected with the drainage receptacle 216 via an air line 224, and a liquid pump 222b located on the supply line 218 that pumps fresh medium from the medium tank 212 to the bioreactor vessel 214.

In operation, the pump 222b is activated to force fresh medium from the medium tank 212 through the supply line 218 and into the upper compartment 240 of the bioreactor vessel 214 through the nozzle 246. The pump 222a is activated to create suction in the drainage receptacle 216 that draws spent (e.g., waste) medium from the lower compartment 242 of the bioreactor vessel 214 through the drainage line 220 and into the drainage receptacle 216.

The employment of two pumps 222a, 222b has the potential benefit of separately controlling the flow of medium into and out of the bioreactor vessel 214. For example, fresh medium may be introduced into the bioreactor vessel 214 via the pump 222a, thereby providing the rinsing action on the plant matter as described above, and allowed to drain from the plant matter and screen 236 prior to draining of some of the spent media with the pump 222b. In some embodiments, two or more cycles of removing media may occur by activating pump 222b prior to activating pump 222a, which may improve removing waste media and/or contaminants from the plant material and/or vessel 214. Additionally, by using separate pumps 222a, 222b, different pumping pressures and/or durations may be employed for the supply and draining actions.

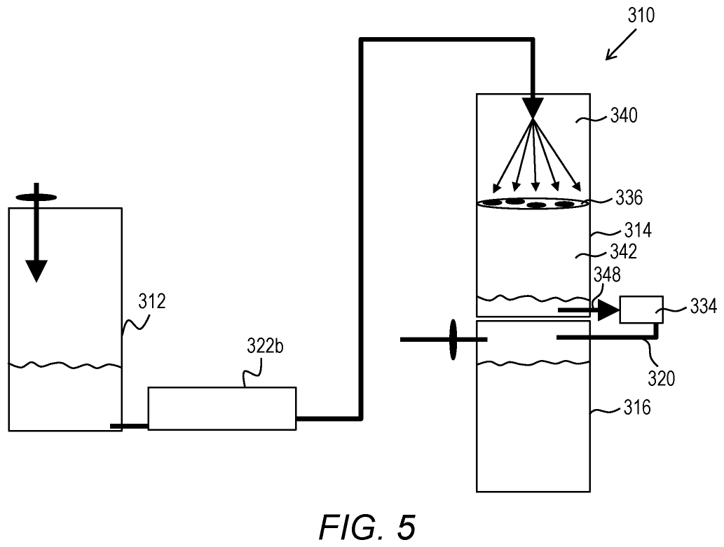

FIG. 5 illustrates a further embodiment of a bioreactor assembly, designated broadly at 310. The bioreactor assembly 310 is similar to the bioreactor assembly 210 described above with the exception that it lacks a pump to provide suction to the drainage receptacle 316. Instead, the drainage receptacle 316 is configured and located so that spent medium exiting the bioreactor vessel 314 through the outlet 348 drains gravimetrically through the check valve 334 and the drainage line 320 to the drainage receptacle 316. Thus, in operation, when fresh medium is pumped via the pump 322b from the medium tank 312 into the upper compartment 340 of the bioreactor vessel 314 and onto the screen 336, the pressure within the bioreactor vessel 314 is sufficient to force the spent medium in the lower compartment 342 through the outlet 348 and check valve 334 into the drainage line 320; once in the drainage line 320, gravity draws the spent medium into the drainage receptacle 316. This arrangement may have the advantage of utilizing a single pump 322b, which may both simplify the system and provide operational flexibility in selecting pump pressure, operation duration and the like.

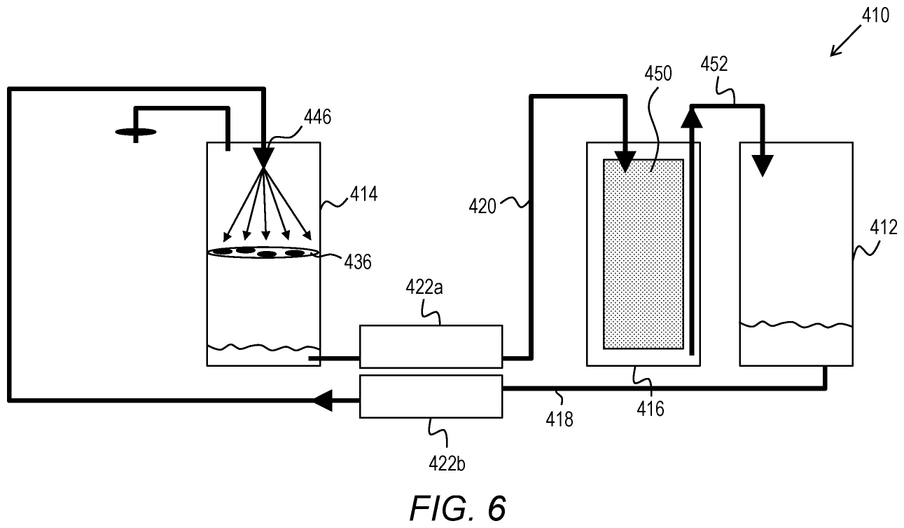

FIG. 6 illustrates another embodiment of a bioreactor assembly, which is designated broadly at 410. The bioreactor assembly 410 has a bioreactor vessel 414 that is fed by a medium tank 412 and that drains into a drainage receptacle 416. However, in this embodiment that drainage receptacle 416 includes a filter 450 that is configured to filter contaminants (such as bacteria and/or fungi) from the spent medium, thereby refreshing the spent medium to a condition usable as fresh medium. FIG. 6 shows that the drainage receptacle 416 and the medium tank 412 are fluidly connected with a replenishment medium line 452. The bioreactor assembly 410 includes a liquid pump 422a on the drainage line 420 between the bioreactor vessel 414 and the drainage receptacle 416, and includes another liquid pump 422b on the supply line 418 between the medium tank 412 and the bioreactor vessel 414.

In operation, when replenishment of medium is needed, the pump 422a pumps spent liquid medium from the bioreactor vessel 414 through the drainage line 420 into the drainage receptacle 416. The pump 422b pumps fresh medium from the medium tank 412 through the supply line 418 and into the nozzle 446 for spraying misting onto the plant matter on the screen 436. Notably, either of the pumps 422a, 422b may also be employed to force spent medium through the filter 450 and into the medium tank 412 through the replenishment medium line 452.

In addition to the potential benefit of recycling spent medium into fresh medium, this arrangement also has the potential benefit of separately controlling the flow of medium into and out of the bioreactor vessel 414. For example, fresh medium may be introduced into the bioreactor vessel 414 via the pump 422a, thereby providing the rinsing action described above, and allowed to drain from the plant matter and screen prior to draining of some of the spent media with the pump 422b. Additionally, by using separate pumps 422a, 422b, different pumping pressures and/or durations may be employed for the supply and draining actions.

It should be noted that any or all of the sensor(s) 52, the timer 54, the controller 56, and the CO₂ line 58 shown in connection with the bioreactor assembly 10 may also be employed with any of the bioreactor assemblies 110, 210, 310, 410. In some embodiments, a bioreactor vessel, medium tank, and/or the drainage receptacle of a bioreactor assembly of the present invention is/are autoclavable and/or configured to be sterilized. In some embodiments, a bioreactor assembly of the present invention is autoclavable and/or configured to be sterilized. In addition, multiple bioreactor vessels may be employed with a single medium tank and/or a single drainage receptacle.

Non-limiting examples of plants and/or plant material thereof (e.g., explants) useful with the present invention include turf grasses (e.g., bluegrass, bentgrass, ryegrass, fescue), feather reed grass, tufted hair grass, miscanthus, arundo, switchgrass, vegetable crops, including artichokes, kohlrabi, arugula, leeks, asparagus, lettuce (e.g., head, leaf, romaine), malanga, melons (e.g., muskmelon, watermelon, crenshaw, honeydew, cantaloupe), cole crops (e.g., brussels sprouts, cabbage, cauliflower, broccoli, collards, kale, Chinese cabbage, bok choy), cardoni, carrots, napa, okra, onions, celery, parsley, chick peas, parsnips, chicory, peppers, potatoes, cucurbits (e.g., marrow, cucumber, zucchini, squash, pumpkin, honeydew melon, watermelon, cantaloupe), radishes, dry bulb onions, rutabaga, eggplant, salsify, escarole, shallots, endive, garlic, spinach, green onions, squash, greens, beet (sugar beet and fodder beet), sweet potatoes, chard, horseradish, tomatoes, turnips, and spices; a fruit crop such as apples, apricots, cherries, nectarines, peaches, pears, plums, prunes, cherry, quince, fig, nuts (e.g., chestnuts, pecans, pistachios, hazelnuts, pistachios, peanuts, walnuts, macadamia nuts, almonds, and the like), citrus (e.g., clementine, kumquat, orange, grapefruit, tangerine, mandarin, lemon, lime, and the like), blueberries, black raspberries, boysenberries, cranberries, currants, gooseberries, loganberries, raspberries, strawberries, blackberries, grapes (wine and table), avocados, bananas, kiwi, persimmons, pomegranate, pineapple, tropical fruits, pomes, melon, mango, papaya, and lychee, a field crop plant such as clover, alfalfa, timothy, evening primrose, meadow foam, corn/maize (field, sweet, popcorn), hops, jojoba, buckwheat, safflower, quinoa, wheat, rice, barley, rye, millet, sorghum, oats, triticale, sorghum, tobacco, kapok, a leguminous plant (beans (e.g., green and dried), lentils, peas, soybeans), an oil plant (rape, canola, mustard, poppy, olive, sunflower, coconut, castor oil plant, cocoa bean, groundnut, oil palm), duckweed, Arabidopsis, a fiber plant (cotton, flax, hemp, jute), Cannabis (e.g., Cannabis sativa, Cannabis indica, and Cannabis ruderalis), lauraceae (cinnamon, camphor), or a plant such as coffee, sugar cane, tea, and natural rubber plants; and/or a bedding plant such as a flowering plant, a cactus, a succulent and/or an ornamental plant (e.g., roses, tulips, violets), as well as trees such as forest trees (broad-leaved trees and evergreens, such as conifers; e.g., elm, ash, oak, maple, fir, spruce, cedar, pine, birch, cypress, eucalyptus, willow), as well as shrubs and other nursery stock. In some embodiments, a method and/or system of the present invention may be used in a method of modifying (e.g., editing) a plant and/or cell thereof (e.g., a plant and/or cell thereof from maize, soybean, wheat, canola, rice, tomato, pepper, sunflower, raspberry, blackberry, black raspberry and/or cherry). In some embodiments, the plant material (e.g., explant) is a blackberry, raspberry (e.g., red raspberry), artic bramble, or cherry plant material. In some embodiments, the plant material is from a plant in the Rubus family.

The invention will now be described with reference to the following examples. It should be appreciated that these examples are not intended to limit the scope of the claims to the invention, but are rather intended to be exemplary of certain embodiments. Any variations in the exemplified methods that occur to the skilled artisan are intended to fall within the scope of the invention.

### EXAMPLES

### Example 1

Blackberry nodes were harvested from greenhouse grown plants and co-cultured with Agrobacterium tumifaciens containing the reporter gene zsGreen for a period of 24 hours. The blackberry tissues were collected and placed on a permeable member in the bioreactor vessel of the bioreactor assembly with a medium tank, bioreactor vessel, drainage receptacle and pump arranged as shown in FIG. 2. The medium tank held media including antibiotics and plant growth hormones. The blackberry tissues were misted for 10 seconds per hour and spent media was removed from the bioreactor vessel every hour. After the first 24 hours of operation, it was observed that the waste medium was yellow, likely indicating that phenolics are washed off the blackberry tissue, which was an added benefit not considered previously.

The foregoing is illustrative of the present invention and is not to be construed as limiting thereof. The invention is defined by the following claims, with equivalents of the claims to be included therein.

That which is claimed is:

1. A method of co-culturing a plant explant and bacteria, the method comprising:
culturing a plant explant comprising (e.g., inoculated with) bacteria in a bioreactor vessel, the bioreactor vessel comprising an internal cavity, a permeable member that divides the internal cavity into upper and lower compartments, a dispensing inlet positioned to feed the upper compartment, and a drainage outlet configured to drain the lower compartment, wherein the plant explant comprising the bacteria is present on the permeable member;
spraying media onto the plant explant comprising the bacteria via the dispensing inlet, wherein, following spaying, excess media travels through the permeable member to the lower compartment to provide waste media in the lower compartment; and
removing the waste media via the drainage outlet, thereby co-culturing the plant explant and the bacteria.

2. The method of claim 1, further comprising, prior to culturing the plant explant comprising the bacteria in the bioreactor vessel, contacting the plant explant and the bacteria (e.g., inoculating the plant explant with the bacteria) to thereby provide the plant explant comprising the bacteria.

3. The method of claim 2, wherein contacting the plant explant and the bacteria occurs outside the bioreactor vessel and the method further comprises, prior to culturing the plant explant comprising the bacteria in the bioreactor vessel, placing the plant explant comprising the bacteria on the permeable member in the bioreactor vessel.

4. The method of claim 2, wherein contacting the plant explant and the bacteria occurs inside the bioreactor vessel and the method further comprises, prior to culturing the plant explant comprising the bacteria in the bioreactor vessel, contacting (e.g., spraying) a composition comprising the bacteria onto the explant via the dispensing inlet to thereby provide the plant explant comprising the bacteria.

5. The method of any preceding claim, wherein the internal cavity of the bioreactor vessel is a sterile environment.

6. The method of any preceding claim, wherein, during the culturing, spraying, and/or removing steps, the bioreactor vessel is present in a closed system.

7. The method of any preceding claim, wherein culturing the plant explant comprising the bacteria in the bioreactor vessel comprises providing temperature and/or light conditions sufficient to maintain and/or grow the plant explant comprising the bacteria.

8. The method of any preceding claim, wherein the dispensing inlet comprises a spray nozzle (e.g., an atomizing nozzle) and spraying the media onto the plant explant comprising the bacteria comprises spraying the media through the spray nozzle and onto the plant explant comprising the bacteria. 9 The method of claim 8, wherein the spray nozzle is configured to produce a mist, atomized spray, and/or aerosol.

10. The method of any preceding claim, wherein spraying the media onto the plant explant comprising the bacteria comprises washing contaminants and/or metabolites (e.g., bacteria and/or phenolics) off the plant explant comprising the bacteria.

11. The method of any preceding claim, wherein spraying the media onto the plant explant comprising the bacteria comprises spraying the media in an amount sufficient to maintain the plant explant comprising the bacteria in a damp state.

12. The method of any preceding claim, further comprising repeating the spraying step one or more times and repeating the removing step one or more times, optionally wherein removing the waste media via the drainage outlet occurs prior to a subsequent spraying step.

13. The method of any preceding claim, wherein spraying the media onto the plant explant comprising the bacteria comprises spraying the media onto the plant explant comprising the bacteria for about 1, 5, or 10 seconds to about 15, 20, or 30 seconds every hour.

14. The method of any preceding claim, wherein spraying the media onto the plant explant comprising the bacteria comprises spraying about 10, 15, or 15 mL to about 20, 25, 30, 40, or 50 mL of media onto the plant explant comprising the bacteria

15. The method of any preceding claim, wherein the method is carried out for about 3, 4, 5, 6, 7, 8, 9, 10, 11, 12, 13, or 14 days, optionally wherein the method is carried out for about 3, 4, 5, 6, 7, 8, 9, 10, 11, 12, 13, or 14 days in a closed system (e.g., without exposing the plant explant comprising the bacteria to the external environment).

16. The method of any preceding claim, further comprising providing a bioreactor assembly that comprises the bioreactor vessel, a medium tank fluidly connected to the dispensing inlet, and a positive pressure source operatively associated with the medium tank and the dispensing inlet, the positive pressure source configured to force the media from the medium tank through the dispensing inlet and into the upper compartment.

17. The method of claim 16, wherein the bioreactor assembly further comprises a drainage receptacle fluidly connected to the drainage outlet, wherein the drainage receptable collects the waste media.

18. The method of claim 16 or 17, further comprising a negative pressure source operatively associated with the drainage outlet configured to draw medium from the lower compartment through the drainage outlet and optionally into the drainage receptacle.

19. The method of any one of claims 16-18, further comprising a fluid line connecting the medium tank and the drainage receptacle.

20. The method of claim 18 or 19, wherein the positive pressure source and the negative pressure source are combined in a single pump unit, and wherein the single pump unit is disposed on the fluid line.

21. The method of claim 20, wherein the single pump unit is configured to pump a gas.

22. The method of any preceding claim, wherein the permeable member comprises a membrane or a screen.

23. The method of any preceding claim, wherein the bioreactor vessel further comprises a sensor positioned to sense an operational parameter of the bioreactor vessel, and the method further comprises sensing the operation parameter of the bioreactor vessel.

24. The method of claim 23, wherein sensing the operation parameter of the bioreactor vessel comprises detecting an operational parameter selected from the group consisting of: humidity, temperature, pressure, oxygen concentration, carbon dioxide concentration, light intensity, day light length, and any combination thereof.

25. The method of any preceding claim, wherein the bioreactor vessel is a first bioreactor vessel and the plant explant comprising the bacteria is a first plant explant comprising first bacteria, and the method further comprises culturing a second plant explant comprising a second bacteria in a second bioreactor vessel, optionally wherein the second bioreactor vessel is fluidly connected to the medium tank and/or to the drainage receptacle.

26. The method of any preceding claim, further comprising supplying carbon dioxide into the internal cavity.

27. The method of any one of claims 16-26, wherein spraying media onto the plant explant comprising the bacteria and removing the waste media occur intermittently and a timer that is operatively connected with the negative pressure source and/or the positive pressure source is configured to induce the negative and/or positive pressure sources to operate intermittently and thereby spray the media and/or remove the waste media intermittently.

28. The method of any one of claims 17-27, wherein the drainage receptacle is positioned below the lower compartment such that waste media from the lower compartment may drain gravimetrically from the lower compartment to the drainage receptacle.

29. The method of any one of claims 16-28, wherein the bioreactor assembly further comprises a filter positioned on the fluid path between the medium tank and dispensing inlet.

30. The method of any preceding claim, wherein the bioreactor vessel further comprises a vent (e.g., a one-way vent), optionally wherein the vent comprises a filter.

31. The method of any preceding claim, wherein the bioreactor vessel, medium tank, and/or the drainage receptacle are autoclavable and/or configured to be sterilized, optionally wherein the bioreactor assembly is autoclavable and/or configured to be sterilized.

32. The method of any preceding claim, wherein culturing the plant explant comprising the bacteria is devoid of culturing the plant explant comprising the bacteria on a solid media (e.g., solid growth media), optionally wherein the bioreactor vessel is devoid of a solid media (e.g., solid growth media).

33. The method of any preceding claim, further comprising recycling the waste media to provide recycled media, optionally wherein recycling the waste media comprises filtering the waste media to provide the recycled media.

34. The method of claim 33, further comprising pumping the recycled media into the medium tank and spraying the recycled media onto the plant explant comprising the bacteria, wherein the positive pressure source is configured to force the recycled media from the medium tank through the dispensing inlet and into the upper compartment.

35. The method of any preceding claim, wherein, following culturing for a period of time (e.g., 7 days) the method increases the survival rate of the plant explants comprising the bacteria compared to a control method (e.g., a method of culturing plant explants comprising bacteria on a solid media) comprising culturing for the same period of time.

36. The method of any preceding claim, wherein the method increases the processing rate (i.e., amount of plant material processed (e.g., cultured) over a period of time) of plant explants comprising bacteria compared to the processing rate of plant explants comprising bacteria for a control method (e.g., a method of culturing plant explants comprising bacteria on a solid media).

37. A bioreactor assembly, comprising:
a bioreactor vessel having an internal cavity and a permeable member that divides the internal cavity into upper and lower compartments, the bioreactor vessel further including a dispensing inlet positioned to feed the upper compartment and a drainage outlet configured to drain the lower compartment;
a medium tank fluidly connected to the dispensing inlet;
a drain fluidly connected to the drainage outlet; and
a positive pressure source operatively associated with the medium tank and the dispensing inlet configured to force medium from the medium tank through the dispensing inlet and into the upper compartment.

38. The bioreactor assembly of claim 37, further comprising a negative pressure source operatively associated with the drain configured to draw medium from the lower compartment through the drainage outlet and into the drain, optionally wherein the drain is fluidly connected to a drainage receptacle and the negative pressure source is configured to draw medium into the drainage receptacle.

39. The bioreactor assembly of claim 37 or 38, further comprising a fluid line connecting the medium tank and the drain and/or drainage receptacle.

40. The bioreactor assembly of claim 39, wherein the positive pressure source and the negative pressure source are combined in a single pump unit, and wherein the single pump unit is disposed on the fluid line.

41. The bioreactor assembly of claim 40, wherein the single pump unit is configured to pump a gas.

42. The bioreactor assembly of any one of claims 37-41, wherein the permeable member comprises a membrane or a screen.

43. The bioreactor assembly of any one of claims 37-42, wherein the dispensing inlet comprises a spray nozzle, optionally wherein the spray nozzle is an atomizing nozzle.

44. The bioreactor assembly of claim 43, wherein the spray nozzle is configured to produce a mist, atomized spray, and/or aerosol.

45. The bioreactor assembly of any one of claims 37-44, further comprising a sensor positioned to sense an operational parameter of the bioreactor vessel.

46. The bioreactor assembly of claim 45, wherein the sensor detects an operational parameter selected from the group consisting of: humidity, temperature, pressure, oxygen concentration, carbon dioxide concentration, light intensity, day light length, and any combination thereof.

47. The bioreactor assembly of any one of claims 37-46, wherein the bioreactor vessel is a first bioreactor vessel, and bioreactor assembly further comprises a second bioreactor vessel that is fluidly connected to the medium tank and/or to the drainage receptacle.
The bioreactor assembly of any one of claims 37-47, wherein the internal cavity has a volume of about 500, 600, 700, 800, or 900 mL to about 1, 1.5, 2, 2.5, 3, 3.5, 4, 4.5, 5, 5.5, 6, 6.5, 7, 7.5, or 8 L.

48. The bioreactor assembly of any one of claims 37-48, further comprising a CO₂ source fluidly connected with the internal cavity.

49. The bioreactor assembly of any one of claims 37-49, further comprising a timer operatively connected with the negative pressure source and/or the positive pressure source, the timer configured to induce the negative and/or positive pressure sources to operate intermittently.

50. The bioreactor assembly of any one of claims 37-50, wherein the drain is positioned below the lower compartment, such that fluid from the lower compartment may drain gravimetrically from the lower compartment to the drain.

51. The bioreactor assembly of any one of claims 37-51, further comprising a filter in fluid communication with the medium tank and dispensing inlet and positioned on the fluid path between the medium tank and dispensing inlet.

52. The bioreactor assembly of any one of claims 37-52, further comprising a vent (e.g., a one-way vent) in the internal cavity, optionally wherein the vent comprises a filter.

53. The bioreactor assembly of any one of claims 37-53, wherein the bioreactor vessel, medium tank, and/or the drainage receptacle are autoclavable and/or configured to be sterilized, optionally wherein the bioreactor assembly is autoclavable and/or configured to be sterilized.
